(19) United States
(12) Patent Application Publication
(10) Pub. No.: US 2024/0200013 A1
(43) Pub. Date: Jun. 20, 2024

(54) CELL CULTURE DEVICE WITH A MECHANICAL STIMULATION FUNCTION

(71) Applicant: COMMISSARIAT A L'ENERGIE ATOMIQUE ET AUX ENERGIES ALTERNATIVES, Paris (FR)

(72) Inventors: Camille LAPORTE, Grenoble cedex 09 (FR); Frédéric REVOL-CAVALIER, Grenoble cedex 09 (FR); Remco DEN DULK, Grenoble cedex 09 (FR); Marie-Line COSNIER, Grenoble cedex 09 (FR)

(73) Assignee: COMMISSARIAT A L'ENERGIE ATOMIQUE ET AUX ENERGIES ALTERNATIVES, Paris (FR)

(21) Appl. No.: 18/539,369

(22) Filed: Dec. 14, 2023

(30) Foreign Application Priority Data
Dec. 15, 2022 (FR) 22 13409

(51) Int. Cl. C12M 1/42; C12M 1/12

(52) U.S. Cl. CPC C12M 35/04; C12M 25/02

(57) ABSTRACT

A cell culture device with a function for mechanically stimulating biological tissues (T), including: a fluidic component in which a well is made which is dug in a “main” direction (X), a first elastically deformable porous membrane, referred to as a culture membrane, integrated into the component and extending transversely to the main direction (X) to close off the well, the culture membrane including an upper face intended to receive a cell culture and an opposite lower face, a first fluidic circuit integrated into the component, including a first cavity made in the well under the culture membrane and intended to receive a culture medium suitable for infusing the biological tissues (T), a second elastically deformable non-porous membrane, referred to as the actuating membrane, integrated into the component.

### TECHNICAL FIELD OF THE INVENTION

The present invention relates to a cell culture device with a mechanical stimulation function.

### PRIOR ART

The organs and tissues of a living being are subjected to various mechanical stimuli such as shear stresses in vessels, interstitial flow, compression or, on the contrary, stretching. The reproduction of these biomechanical stresses in microsystems, as well as the integration of the cell microenvironment, is crucial for establishing a physiological and functional organ-on-chip model.

Organs-on-chips (OOCs) enable the complexity of the three-dimensional organization and the chemical and mechanical stimuli specific to a particular organ to be recreated in vitro. An OOC is a microfluidic platform designed to host and maintain in culture a multicellular biological object mimicking the physiological 3D architecture of a tissue or organ. This platform enables the biological object's microenvironment to be controlled, notably by providing a vascular-type infusion.

The mechanical stimulation function of a skin-on-a-chip model appears to be particularly relevant given the limitations of current reconstructed skin models. Current skin models fail to reproduce the skin's permeability parameters and therefore its functionality. In vitro skins are much more permeable to various molecules, which hinders their use in penetration, irritation and sensitization tests for medicinal and cosmetic products.

Patent application WO 2018/096054A1 describes an elastic double membrane system for mimicking diaphragm movements and lung expansion/contraction cycles. The system uses a micro-perforated culture membrane to infuse the stimulated tissues and an actuating membrane that indirectly acts on the culture membrane via the culture medium by increasing the pressure in the circuit used for the culture medium.

However, this solution has a number of drawbacks:
The risk of detachment of tissues subjected to the mechanical stimulation;
The risk of fluid (liquid or gas) leaking through the actuating membrane;
The mechanical limitations of the PDMS material used to produce the culture membrane.

The aim of the invention is to propose a stimulation device that is suitable for overcoming the drawbacks of the prior art.

### DISCLOSURE OF THE INVENTION

This aim is achieved by a cell culture device with a function for mechanically stimulating biological tissues, comprising:
A fluidic component in which a well is made which is dug in a “main” direction,
A first elastically deformable porous membrane, referred to as a culture membrane, integrated into said component and extending transversely to said main direction to close off said well, said culture membrane comprising an upper face intended to receive a cell culture and an opposite lower face, A first fluidic circuit integrated into said component, comprising a first cavity made in said well under said culture membrane, intended to receive a culture medium suitable for infusing said biological tissues,
A second elastically deformable non-porous membrane, referred to as the actuating membrane, integrated into said component,
Means for actuating said actuating membrane,
The actuating membrane being arranged to tightly seal the bottom of said first cavity, and configured to deform under the action of said actuating means to a position in which it mechanically pushes said culture membrane to deform it.

According to an advantageous embodiment, the culture membrane is a three-dimensional foam of constant thickness in the main direction.

According to a particular feature, the three-dimensional foam is structured on its upper face so as to have variations in thickness in the main direction.

According to another particular feature, the foam is made of a material chosen from polyethylene, polyurethane, polyvinyl chloride and rubber.

According to another embodiment, the culture membrane is a perforated strip of material arranged transversely to said main direction. The membrane is then for example made of PDMS.

According to a particular feature, the means for actuating the actuating membrane include a second actuating fluidic circuit, integrated into said component and comprising at least one second pressurization cavity located under the actuating membrane, intended to receive a fluid.

According to another particular feature, the fluid is a liquid or a gas.

The invention also relates to a cell culture system which includes several cell culture devices with a mechanical stimulation function as defined above, said devices being juxtaposed within the same fluidic component.

### BRIEF DESCRIPTION OF THE FIGURES

Further features and advantages will become apparent in the following detailed description, in connection with the figures below:

FIGS. 1A to 1C show a first embodiment example of the device of the invention, respectively before stimulation, with the membranes in the resting state and in the deployed state;

FIGS. 2A to 2C show a second embodiment example of the device of the invention, respectively before stimulation, with the membranes in the resting state and in the deployed state;

FIGS. 3A to 3C show a third embodiment example of the device of the invention, respectively before stimulation, with the membranes in the resting state and in the deployed state;

FIG. 4 shows an advantageous solution for a system including several juxtaposed devices within the same component.

### DETAILED DESCRIPTION OF AT LEAST ONE EMBODIMENT

The device of the invention aims to enable mechanical stimulation of tissues infused with a culture medium.

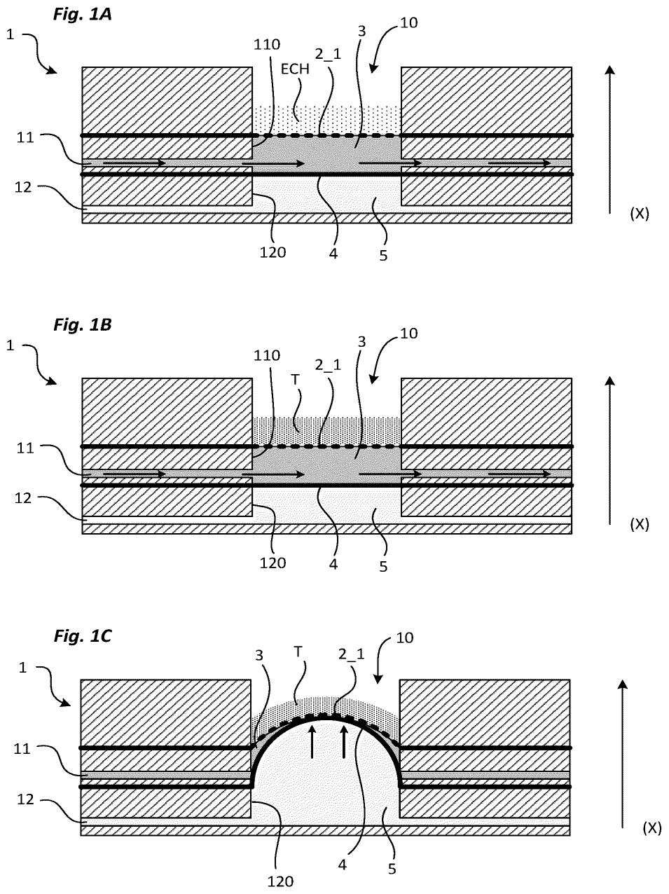

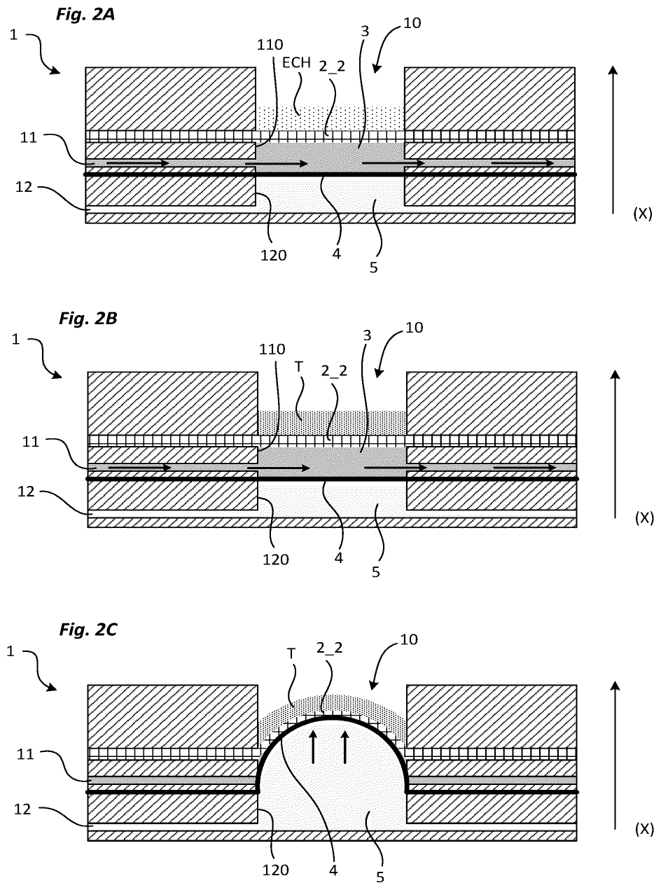

The invention is first described below in relation to FIG. 1A, FIG. 1B and FIG. 1C, and in relation to FIG. 2A, FIG. 2B and FIG. 2C.

The device includes a fluidic component 1. This fluidic component 1 may be made by assembling several layers. It may be made of a material such as, for instance, COC (Cyclic Olefin Copolymer), glass or PMMA (Polymethyl Methacrylate).

The various layers of component 1 are assembled in a direction, referred to hereinbelow as the main direction (X), oriented towards the top of the sheet. This main direction (X) is vertical when the component is placed on a support. The terms “upper”, “lower”, “top”, “bottom”, “above” and “below” are to be understood in relation to this main direction.

A well 10 is dug in said component 1, along said main direction (X), so as to pass through several layers of the component. The well 10 is open on the upper side of component 1 and closed on the lower side of the component.

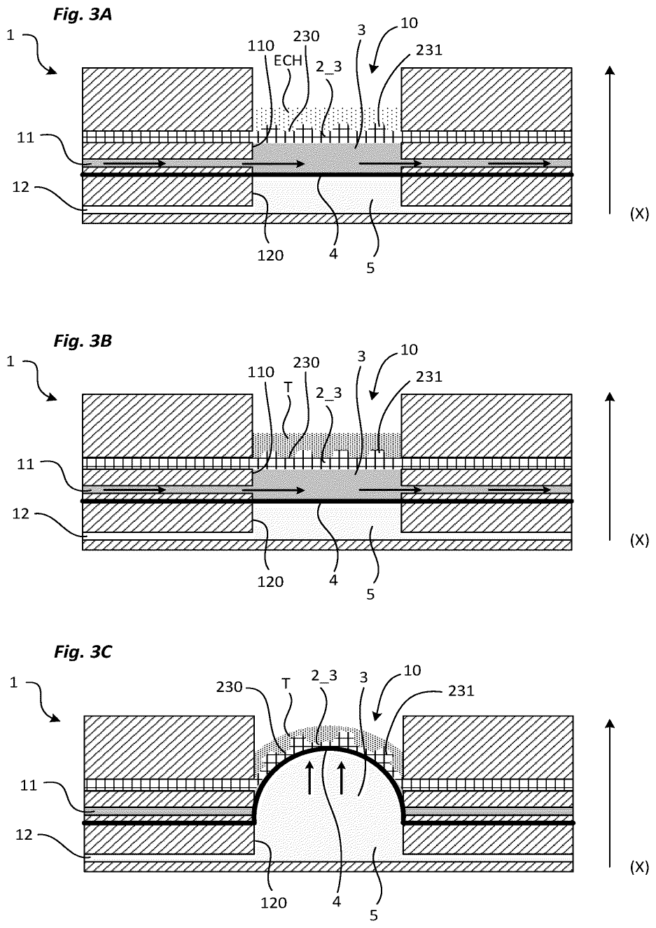

The device includes a first deformable porous membrane 2_1, 2_2, integrated into component 1, which seals the well 10 on the upper side, extending transversely to the main direction (X), while leaving a deposition space above it. This membrane 2_1, 2_2 is the culture membrane, i.e. it is where the cell culture is located. It thus includes an upper face accessible from the top of well 10 and an opposite lower face. Its upper face is intended to receive a cell culture (cell culture sample ECH initially deposited in liquid or gel form in well 10, as represented in FIG. 1A, FIG. 2A and FIG. 3A) to be stimulated and infused. The biological tissues T obtained after culture are intended to be mechanically stimulated by means of the device.

This culture membrane 2_1, 2_2 is deformable. In its resting state, it has a flat shape, and in a deployed state, it is able to expand or swell upwards towards the outside of the well.

In FIGS. 1A to 1C, this culture membrane 2_1 may be a strip of material made in two dimensions (i.e. with a very small thickness relative to its other dimensions) and perforated so that a culture medium M required for infusion of the tissues T supported by its upper surface can pass through it. In this configuration, the membrane may be made of a material such as PDMS (PolyDiMethylSiloxane).

Preferentially, as shown in FIGS. 2A to 2C, the culture membrane 2_2 used is a three-dimensional porous foam. It thus includes one or more superimposed rows of open cells. Its porosity allows the cells of the cultured tissues T to anchor themselves in the pores of the foam, thus preventing the tissues T from detaching during mechanical stimulation. In this embodiment, the foam forming the culture membrane 2_2 has a constant thickness in the main direction. It is cut in the other two directions to fit into the component and close off the cavity at the top.

In a non-limiting manner, this foam may be made of a material chosen from polyethylene (PE), polyurethane (PU), polyvinyl chloride (PVC), rubber or equivalent materials. By way of example, polyethylene foam allows considerable elastic deformation (more than 100% elasticity), while having sufficient porosity to allow the culture medium to pass through.

The foam also has the advantage that it can be manufactured very easily, with the required porosity and dimensions suited to the mechanical stimulation device.

The use of a foam affords the following advantages:
It may be used as a reservoir for media or various reagents;
Its porosity can be easily chosen and not necessarily uniform throughout its volume;
It may be easily manufactured at low cost using biocompatible materials;
It acts as an attachment zone for the biological tissues to be stimulated. Specifically, the use of a three-dimensional porous membrane (a foam) allows cells to proliferate partly inside the foam, which enables better attachment of cellular tissue during mechanical deformation.

Component 1 also includes a first fluidic circuit 11 having a first cavity 110 made in well 10, under the culture membrane 2_1, 2_2. This first fluidic circuit 11 is dedicated to the circulation of the culture medium 3, injected in liquid form inside the first cavity 110. The lower face of the culture membrane 2_1, 2_2 thus comes into contact with the culture medium 3 present in the cavity 110. The culture medium 3 may diffuse through the culture membrane 2_1, 2_2 to infuse the tissues T deposited on the upper face of the culture membrane 2_1, 2_2.

The device also includes a second deformable non-porous membrane 4, referred to as the actuating membrane 4. This actuating membrane 4 is integrated into component 1 so as to tightly seal the first cavity 110 from below, and thus form the bottom of said first cavity 110.

It may be made of a highly elastic material, for example a silicone-based material (for example an Ecoflex membrane—registered trademark). Such a membrane 4 is capable of elastic deformation (to more than 100% relative to its initial configuration) without risk of rupturing and without compromising the leaktightness of the device.

According to a particular aspect of the invention, the actuating membrane 4 is actuated by means of a second fluidic circuit 12, advantageously integrated into the component 1. This second fluidic circuit 12 includes, for example, a second cavity 120 made in component 1, below the actuating membrane 4. A fluid 5 may thus be injected into said second cavity 120, an increase in the pressure of the fluid 5 in the second cavity 120 causing an upward deformation of the actuating membrane 4.

The culture membrane 2_1, 2_2 and the actuating membrane 4 are located on the same axis, parallel to the main direction X, and are both arranged transversely to cover the cross-section of the well 10 at two different heights. They are located one above the other, with the actuating membrane 4 positioned below the culture membrane 2_1, 2_2, separated from the latter by the first cavity 110 (FIG. 1B and FIG. 2B). The culture membrane 2_1, 2_2 closes the first cavity 110 from above and the actuating membrane 4 closes the first cavity 110 from below. The first cavity 110 is located between the two membranes.

According to another aspect of the invention, the deformation of the culture membrane 2_1, 2_2 is obtained by exerting a direct mechanical stress on the actuating membrane 4. In other words, the actuating membrane 4 is deformed sufficiently to come into physical contact with the culture membrane 2_1, 2_2 and push it so as to deform it. The deformation of the culture membrane 2_1, 2_2 is then transmitted to the biological tissues T placed on its upper face (FIG. 1C and FIG. 2C). The deformation of the actuating membrane 4 is made possible by the presence of the first cavity 110 in which the culture medium 3 is placed. As the actuating membrane 4 pushes the culture membrane 2_1, 2_2 as it expands into the first cavity 110, the volume occupied by the culture medium 3 in the first cavity 110 is smaller and may be virtually zero if the deformed actuating membrane 4 follows the shape of the lower face of the culture membrane 2_1, 2_2. To enable the culture medium 3 to be evacuated during actuation, and to enable the actuating membrane 4 to push the culture membrane 2_1, 2_2 and come into physical contact therewith, it is necessary to provide at least one channel for evacuating the culture medium in the first fluidic circuit 11, to the outside of the first cavity 110.

Advantageously, the fluid 5 injected into this second fluidic circuit 12 is a non-compressible liquid. The use of a liquid makes it possible to better regulate the deformation of the actuating membrane 4 and therefore the mechanical stimulation applied to the tissues T, via the culture membrane 2_1, 2_2. Needless to say, it would also be possible to use a gas to perform a pneumatic actuation.

It should be noted that the mechanical deformation applied during cultivation is a cyclic deformation of the order of a few deformations per minute to a few deformations per second. By way of example, the amplitude of the deformation is of the order of 5% to 50%, performed precisely and reproducibly from one cycle to the next.

The device of the invention thus uses a two-membrane mechanism: a porous membrane and a non-porous membrane. This double-membrane principle has a number of advantages, including:
A controlled and reproducible mechanical deformation, notably by virtue of the use of actuation by means of a non-compressible liquid;
An actuation solution independent of the culture of the biological tissues T;
A solution that limits the risk of biological tissue T detachment during stimulation.

An embodiment variant is described below in relation to FIGS. 3A to 3C. In this variant, the references used for the preceding embodiments are retained insofar as the elements are identical.

FIG. 3A illustrates the deposition of cell culture ECH in well 10 of fluidic component 1.

According to this variant, the three-dimensional foam used to make the culture membrane 2_3 is structured. The term “structuring” means that the foam may include variations in thickness along the main direction (X) (FIG. 3B). These variations in thickness are materialized for example by hollows 230 or spikes or studs 231 on its upper face. When the culture membrane 2_3 is deformed (FIG. 3C), by direct mechanical stress from the actuating membrane 4, the structuring of its upper face makes it possible to apply different levels of stretching to the stimulated biological tissues T.

The other features and advantages of the solutions described above remain valid for this last embodiment.

It should be noted that conclusive tests were conducted to ensure that biological tissues T deposited on a foam can indeed be infused through the foam by a culture medium.

It should be noted that each fluidic circuit 11, 12 includes valves for controlling the supply of fluid to the first cavity 110 and the second cavity 120. This valve mechanism, associated with the actuating membrane 4, may notably be used to operate the device as a pump, enabling the culture medium 3 to be renewed:
A first valve is thus placed upstream of the first cavity 110 and a second valve downstream of the first cavity 110.
During mechanical stimulation, the first valve is closed and the second valve is opened. The deformation of the actuating membrane 4 then pushes the culture medium 3 downstream, mechanically pushing the culture membrane 2_1, 2_2.
The second valve is closed. The actuating membrane 4 is still in its deployed state.
The first valve is then opened. The stress on the actuating membrane 4 is then removed. On returning to the resting state, the actuating membrane 4 pumps the liquid of the culture medium 3 into the first cavity 110. The culture medium is thus easily renewed.

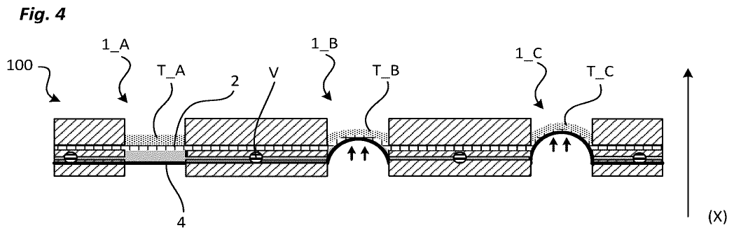

Advantageously, as illustrated by FIG. 4, the device of the invention may be duplicated, for example within the same component, with several juxtaposed devices 1_A, 1_B, 1_C, which makes it possible to obtain a complete system 100. A single actuating membrane 4 and a single culture membrane 2, common to all the devices, may thus be used. The culture membrane is, for example, the foam used in the variant shown in FIGS. 2A to 2C. It may also be structured, at least partially, for example on a single device among all the juxtaposed devices. Valves V are placed, for example, on the first fluidic circuit 11 to control the supply of culture medium 3. Independent actuation of the actuating membrane 4 at each device may be provided so as optionally to be able to ensure different stimulation levels from one device to another.

By means of this system, it is thus possible (the options below may be combined together):
To place different samples from one device to another;
To use different foams from one device to another, which foams may have various porosities and various structurings;
To apply different stimuli from one device to another;
To apply different growing conditions from one device to another;
To apply different chemical treatments from one device to another;

In FIG. 4, the biological tissues T_A, T_B, T_C may be mechanically stimulated separately in each device 1_A, 1_B, 1_C of the system 100.

The invention described above will notably be very relevant for mechanical stimulation of the skin. It will also be possible to use it for a pulmonary or intestinal model.

1. A cell culture device with a function for the mechanical stimulation of biological tissues (T), comprising:
a fluidic component wherein a well is made which is dug in a “main” direction (X),
a first elastically deformable porous membrane, referred to as a culture membrane, integrated into said component and extending transversely to said main direction (X) to close off said well, said culture membrane comprising an upper face intended to receive a cell culture and an opposite lower face,
a first fluidic circuit integrated into said component, comprising a first cavity made in said well under said culture membrane and intended to receive a culture medium suitable for infusing said biological tissues (T),
a second elastically deformable non-porous membrane, referred to as the actuating membrane, integrated into said component,
means for actuating said actuating membrane,
wherein:
the actuating membrane is arranged to tightly seal the bottom of said first cavity, and configured to deform under the action of said actuating means to a position wherein said actuating membrane mechanically pushes said culture membrane to deform it.

2. The device according to claim 1, wherein the culture membrane is a three-dimensional foam of constant thickness in the main direction (X).

3. The device according to claim 2, wherein the three-dimensional foam is structured on its upper face so as to have variations in thickness in the main direction (X).

4. The device according to claim 2, wherein the foam is made of a material chosen from polyethylene, polyurethane, polyvinyl chloride and rubber.

5. The device according to claim 1, wherein the culture membrane is a perforated strip of material arranged transversely to said main direction.

6. The device according to claim 5, wherein the membrane is made of PDMS.

7. The device according to claim 1, wherein the means for actuating the actuating membrane include a second actuating fluidic circuit, integrated into said component and comprising at least one second pressurization cavity located under the actuating membrane and intended to receive a fluid.

8. A cell culture system, comprising several cell culture devices with a mechanical stimulation function as defined in claim 1, said devices being juxtaposed within the same fluidic component.
